(19) United States
(12) Patent Application Publication
(10) Pub. No.: US 2011/0093056 A1
(43) Pub. Date: Apr. 21, 2011

(54) Use of Plasma in Formation of Biodegradable Stent Coating

(75) Inventors: Stephen L. Kaplan, San Carlos, CA (US); Patrick H. Ruane, Redwood City, CA (US); Eric A. Lang, Foster City, CA (US); Torsten Kimura, Belmont, CA (US)

(73) Assignee: Xtent, Inc., Menlo Park, CA (US)

(21) Appl. No.: 12/977,472

(22) Filed: Dec. 23, 2010

Related U.S. Application Data
(63) Continuation of application No. 11/757,093, filed on Jun. 1, 2007.
(60) Provisional application No. 60/810,522, filed on Jun. 2, 2006.

(51) Int. Cl. A61F 2/84; C23C 16/50; A61F 2/82

(52) U.S. Cl. 623/1.11; 427/2.21; 623/1.46

(57) ABSTRACT

Metallic stents are treated with a gaseous species in a plasma state under conditions causing the species to polymerize and to be deposited in polymerized form on the metallic stent surface prior to the application of a drug-polymer mixture, which is done by conventional non-plasma deposition methods. The drug-polymer mixture once applied forms a coating on the stent surface that releases the drug in a time-release manner and gradually erodes, leaving only the underlying plasma-deposited polymer. In certain cases, the plasma-deposited polymer itself erodes or dissolves into the physiological medium over an extended period of time, leaving only the metallic stent. While the various polymers and drug remain on the stent, the plasma-deposited polymer enhances the adhesion of the drug-polymer anchor coating and maintains the coating intact upon exposure to the mechanical stresses encountered during stent deployment.

### CROSS-REFERENCES TO RELATED APPLICATIONS

The present application is a continuation of U.S. patent application Ser. No. 11/757,093 (Attorney Docket No. 021629-003910US) filed Jun. 1, 2007, which is a non-provisional of, and claims the benefit of U.S. Provisional Application No. 60/810,522 (Attorney Docket No. 021629-003900US), filed Jun. 2, 2006, the full disclosures of which are incorporated herein by reference.

### BACKGROUND OF THE INVENTION

1. Field of the Invention

This invention resides in the field of medical devices and methods and more specifically in the field of vascular catheters and stents that incorporate therapeutic or otherwise bioactive materials.

2. Description of the Background Art

As is well known among clinicians experienced in the treatment of coronary heart disease, the early use of angioplasty for the opening of blood vessels obstructed by stenotic lesions was plagued by frequent restenosis, the tendency of obstructions to re-form during the months following the procedure. Restenosis is thought to be a response of the vascular tissue to the trauma caused by the mechanical action of the devices used in angioplasty, notably angioplasty balloons, pressing against the lesions to forcibly restore vessel patency. The use of stents has since been introduced to address the restenosis problem. While stents have succeeded considerably in reducing the rate of restenosis, they have not eliminated restenosis entirely. Further reduction in restenosis rates has been achieved by the introduction of drug-eluting stents which add a therapeutic effect to the mechanical effect of the stent. The development of drug-eluting stents has extended beyond merely treating restenosis and now provides localized treatment of a variety of conditions in physiological passageways by delivering therapeutic or bio-active agents directly to sites of interest where the agents can produce a range of beneficial physiological effects. Nevertheless, the most prominent use of drug-eluting stents, together with the elimination or reduction of restenosis, is in the treatment of coronary and peripheral artery disease.

A drug-eluting stent is a stent that contains a bio-active agent applied either to the entire stent surface or to discrete reservoirs or portions of the surface in a manner that causes the stent to release the agent in a continuous and sustained release profile into the physiological environment. Since a wide range of bio-active agents has been disclosed for delivery by stents, the term “drug” is used herein for convenience to represent these agents in general. The drug can be applied to the stent by itself or suspended in a matrix, and the matrix can be either durable or erodible. When the drug is suspended in a matrix, the sustained-release effect is achieved either by allowing the physiological fluid to diffuse into the matrix, dissolve the drug, and diffuse out again with the dissolved drug, or, in the case of erodible matrices, by continuously exposing fresh drug due to the erosion of the matrix, or by a combination of diffusion and erosion. The period of time over which the drug is released by either mechanism is controlled by the chemical properties of the matrix including its solubility or erodibility, the nature and strength of any attraction between the matrix and the drug, and the physical form of the matrix including its porosity and thickness, and the drug loading. Restenosis prevention, and most physiological conditions that are treatable in this manner, respond best to drug administration over a designated but limited period of time. Continued retention of the drug, the matrix, or both beyond this period of time is both unnecessary and potentially detrimental to the surrounding tissue and the health of the subject. The optimal drug-eluting stent for any particular physiological condition is therefore one that fully expels both drug and matrix, and in general all components other than the underlying stent itself, shortly after the desired treatment period which may last from a few hours to several weeks or several months, depending on the condition.

An additional consideration in the construction and formulation of drug-eluting stents is the integrity of the coating and its ability to remain intact during deployment of the stent. The typical stent is a tubular structure, often with a mesh or lattice-type wall. Stent delivery techniques are well known in the art and in general the tubular structure is maintained in a compressed configuration during insertion into the body, and once it reaches the location of the obstruction, often the site of a stenotic lesion in an artery, the stent is expanded to remove the obstruction. In its compressed configuration, the stent can be guided to and inserted within the obstructed area, and expansion is achieved either by simply releasing the stent from a size-restricting delivery catheter once the desired location is reached, or by allowing the stent to expand by equilibration to the temperature of the surrounding tissues, or by forcibly expanding the stent by mechanical means. A stent that can be expanded by release from a delivery catheter is a resilient stent that is in a stressed state when restricted by the catheter and a relaxed state when released. A stent that is expanded by equilibration to physiological temperature is one that is made of a shape-memory alloy such as Nitinol. Both types are self-expanding stents. For stents that are expanded only by the application of a force from within the stent interior, the force is typically created by a balloon similar to angioplasty balloons, and the stent is mounted to the balloon in a contracted or “crimped” configuration. In all of these different means of expansion, the stent undergoes a physical deformation and stress during expansion due to bending, changes in curvature, and changes in the angles of stent structural features. The stresses imposed on the coating during these transformations render the coating susceptible to breakage, separation from the stent, or both. Also, in some delivery systems, the stent is placed on the tip of a long catheter and is uncovered and exposed during insertion. As the catheter enters the curved and branched sections of the vascular system, the exposed stent contacts the walls of the blood vessels, which may have hard and rough calcified regions, as well as narrow lesions. Such contact can damage, separate, or remove the coating from the stent. Stent coatings can also be damaged by interactions with components of the delivery catheter.

Coating integrity and strong adhesion to the stent have been achieved in the prior art by the use of a primer layer applied to the stent surface prior to formation of the matrix-supported drug coating. The primer is typically a polymer other than the polymer used as the drug matrix, and a commonly used primer material is parylene (dichloro-p-xylene) in its various forms (i.e., parylene C, N, or HT, or combinations), applied to the stent by vapor deposition. To be effective, the primer layer is generally comparable in thickness to the drug-matrix coating, or within the same order of magnitude, but the primer is typically not biodegradable or erodible, or is substantially less so than the polymeric matrix supporting the drug. The primer thus remains on the stent surface long after the drug and matrix have left the stent. No longer serving a useful function, the residual primer presents a risk of producing an undesirable physiological response in the contacting tissue.

It is therefore desirable to provide stents with a therapeutic agent wherein the stent may be used to deliver the therapeutic agent to a treatment site over a controlled period of time. It is further desired that once the drug has eluted into the treatment site that only the bare metal stent surface remains, or an ultra thin layer of material that does not produce any adverse biocompatibility issues at the treatment site. It is also desirable to provide methods for coupling the therapeutic agent with the stent so that the therapeutic agent remains coupled to the stent during delivery and expansion of the stent.

### BRIEF SUMMARY OF THE INVENTION

It has now been discovered that a drug, preferably one that is matrix-supported, can be deposited on a metallic stent surface without the need for primers of the prior art, or for a primer in general, while still producing a coating that will retain its integrity as the stent is delivered and deployed. This is achieved by first exposing the stent surface to a gaseous species in the presence of a gaseous plasma that will cause the species to polymerize on the surface of the stent and enhance adhesion of the drug coating. While not intending to be bound by any particular theory, it is believed that the plasma-deposited polymer may enhance drug adhesion by either interacting with (i.e., bonding to, grafting to, or adhering to by some other mechanism) the overlying drug, the matrix in the case of a matrix-supported drug, or the underlying stent, by forming an ultra-thin tie layer. The ultra-thin tie layer preferably ranges in thickness from about 100 Å to about 5,000 Å, more preferably from about 100 Å to about 1,000 Å and even more preferably from about 100 Å to 500 Å. In some cases, the tie layer may be a single molecule in thickness, while in other cases the layer may be several molecules in thickness, depending on the type and degree of polymerization. In one aspect of the invention, the tie layer formed by the plasma-deposited polymer on the stent surface is about 500 Å or less in thickness. The drug is then applied, either by itself or as a mixture with a second polymeric material, to the plasma-deposited polymer by conventional techniques other than plasma deposition to achieve a combined coating having a thickness in the micron or mil (thousandths of an inch) range. The ratio of therapeutic agent to polymer in the matrix can vary widely. In preferred embodiments, the percentage by weight of therapeutic agent in the polymer matrix ranges from about 0.1% to 50%, preferably from about 0.1% to about 10% and more preferably from about 0.1% to about 1%. Additionally, the thickness of the polymer matrix often ranges from about 0.2 μm up to about 5 μm.

In embodiments in which a second polymer is included as a matrix for the drug, the second polymer can be either durable (i.e., non-erodible) or bioerodible. Optimal polymers for use as the second polymer and the plasma-deposited polymer will be those that are sufficiently compatible to permit diffusion of the second polymer into the plasma deposited polymer, and possibly to permit bonding of the two layers creating an interpenetrating polymer network. This interpenetrating network does not need to be complete, several molecular layers would be sufficient to establish excellent bonding of the two different layers. The plasma intensity used in forming the initial plasma-deposited polymeric layer will be great enough to cause the polymerizing species to form a flexible and resilient polymer anchor coating yet not so great as to cause crosslinking of the polymer to a degree that renders the initial layer brittle in relation to the expandable stent. While not bound by any theory the judicious selection of plasma parameters can control the plasma polymer's apparent molecular weight (chain extension), crosslink density, swell, modulus and other essential properties such that the plasma deposited layer may act as a modulus gradient or even modulus trough between that of the metal and the drug infused layer thereby reducing the stress on the drug infused layer. Once the second polymer and drug are deposited, the resulting final coating on the stent surface is sufficiently elastic and flexible to withstand the stresses imposed during the deployment of the stent, notably the expansion, stretching, and bending cited above, without producing excessive cracks in the coating or causing the coating to separate from the stent itself. In preferred embodiments, the final coating is sufficiently porous or absorptive of physiological fluid to admit the fluid into the coating where the fluid can dissolve the drug and diffuse outward with the dissolved drug, or in the case of erodible matrices, where the fluid can promote the erosion of the coating. In this manner, the drug is released to the physiological environment in a controlled and sustained manner so as to have its desired therapeutic or bio-active effect. Preferably, the plasma intensity in the initial deposition will also be sufficiently limited to allow the plasma-deposited polymer to swell upon contact with the coating solution of the drug and second polymer to thereby enhance the degree of diffusion of the coating solution into the plasma-deposited polymer, and thereby form an interpenetrating network. As in the prior art, the polymer applied in combination with the drug in the second stage of the deposition erodes in the physiological environment over prolonged exposure to the physiological tissue or fluid. Thus, typically the drug polymer matrix completely erodes away leaving behind an ultra thin plasma polymerized tie layer or anchor coating on the stent. It is more preferable however, if the entire finished coating, including the drug polymer matrix and plasma-deposited polymer, erodes in this manner. Thus, after an extended period of time, the drug and, in the case of bioerodible matrices, the matrix will have been released from the stent, and the stent will contain no polymer at all or at most an extremely thin layer of the plasma-deposited coating, i.e., a substantially monomolecular layer or a layer at most about 500 Å in thickness, with no other residual material. Upon release of the entire drug and erosion of the matrix polymer, an uncoated, or essentially uncoated, stent surface will remain, so that the body fluids and tissues are exposed only to the material of the stent itself. In the case of a durable matrix rather one that is bioerodible, an advantage of the present invention is its elimination of the need for parylene as a primer coating. This advantage is of value in situations where the use of parylene is undesirable.

In preferred embodiments, the invention resides in a stent with a plasma-polymer treated surface, a bioerodible matrix deposited on the plasma-treated surface, and a drug suspended in the matrix. As noted above, the stent is preferably one in which, if any material remains on the stent surface upon full release of the drug, such residual material is at most about 500 Å in thickness. This invention also resides in methods of use, including a method of treating restenosis, of drug delivery, or both, by implanting a stent with a drug coating that leaves at most about 500 Å of residual material on the stent surface after all drug has been released, or a stent in which the stent surface is free of substantially all material typically within 24 months, preferably within 12 months and more preferably within 3-9 months of deployment.

In a first aspect of the present invention a method manufacturing an intraluminal device bearing a therapeutic agent releasable from the device in a time-controlled manner comprises exposing a metallic substrate to a gaseous plasma form of a substance that polymerizes in the plasma form under conditions causing the substance to form a polymer anchor coating of about 500 Å in thickness or less on the substrate. A layer containing the therapeutic agent may then be deposited over the polymer anchor coating. All of the therapeutic agent is substantially releasable into a physiological environment gradually over a period ranging from about one hour up to about six months.

In another aspect of the present invention, a method for manufacturing an intraluminal device bearing a therapeutic agent releasable from the device in a time-controlled manner comprises exposing a metallic substrate to a gaseous plasma form of a substance that polymerizes in the plasma form under conditions causing the substance to form a polymer anchor coating on the substrate. A layer containing the therapeutic agent is then deposited over the anchor coating. The therapeutic agent may be in a polymer matrix that releases substantially all of the therapeutic agent into a physiological environment gradually over a period ranging from about one hour up to about six months and following release of the therapeutic agent, any polymer remaining on the substrate is about 500 Å or less in thickness.

In still another aspect of the present invention, a stent for placement in a body lumen comprises a plurality of struts coupled together forming a substantially tubular structure. The plurality of struts have a polymer anchor coating of about 500 Å in thickness or less disposed thereon and a layer containing a therapeutic agent is positioned over the polymer anchor coating. The polymer anchor coating is formed from a gaseous plasma form of a substance that polymerizes on the struts while in the plasma form, and substantially all of the therapeutic agent releases into a physiological environment gradually over a period ranging from about one hour up to about six months. Sometimes the tubular structure is self-expanding and other times it may be expanded with a balloon. Often the struts are a metal, such as a material like stainless steel, nickel-titanium alloy or cobalt-chromium alloy. The struts may also be a polymer and can be at least partially bioerodible.

In another aspect of the present invention, a method for delivering a therapeutic agent to a target treatment site comprises introducing a delivery catheter having a stent disposed thereon to the target treatment site and deploying the stent into the target treatment site. The stent comprises a plurality of struts having a polymer anchor coating of about 500 Å in thickness or less disposed thereon and a layer containing the therapeutic agent is positioned over the polymer anchor coating. The polymer anchor coating is formed from a gaseous plasma form of a substance that polymerizes on the struts while in the plasma form and substantially all of the therapeutic agent is released into the target treatment site gradually over a period ranging from about one hour up to about 6 months. Often deploying the stent comprises radially expanding the stent into a coronary or peripheral artery where the therapeutic agent inhibits restenosis.

Usually, the polymer anchor coating can withstand significant cracking during expansion and the coating also remains coupled to the intraluminal device without substantially separating from the device during its expansion. Sometimes the polymer anchor coating is continuous over substantially all of a surface of the metallic substrate or stent struts, which may be a material selected from the group consisting of stainless steel, nickel-titanium alloys and cobalt-chromium alloys.

Sometimes the polymer anchor swells when the therapeutic agent is deposited over the polymer anchor and this enhances diffusion of the therapeutic agent into the polymer coating. Often, the substance used to form the polymer anchor is either in gaseous form under ambient conditions or the substance can be volatized. Common materials that may be used for the polymer anchor include but are not limited to materials selected from the group consisting of allyl substituted compounds, acrylic acids, methacrylic acids, acrylates, methacrylates, ethylene glycol, organosilicones, thiophenes, vinyl benzene, vinyl pyrrolidinone and methane.

The substrate may be cleaned prior to plasma polymerization. Plasma processes using non-polymerizable (carbonless) gases such as nitrogen, argon, oxygen, hydrogen, nitrous oxide and many others are very effective in providing atomic level cleanliness and may be incorporated typically as a first step in a multi-step plasma polymerization process. An inert noble gas may also be used during the step of exposing the metallic substrate in order to provide a diluent in the presence of the substance to be polymerized. Masking can be used to cover a portion of the substrate so as to selectively apply the polymer anchor coating to the substrate. The degree of polymerization and cross-linking of the polymer anchor may also be controlled by adjusting operating parameters such as power level and exposure time as well as by applying power in a pulsewise manner. Pulse may be controlled by adjusting pulse frequency, duty cycle and power.

The therapeutic agent may be deposited on to the polymer anchor coating by a number of methods such as dipping, spraying, brush coating, syringe deposition, chemical vapor deposition or plasma deposition. Often, the intraluminal devices or stents are loaded onto a mandrel and rotated during deposition.

Often the therapeutic agent inhibits restenosis. The therapeutic agent may also be at least one of antibiotics, thrombolytics, anti-platelet agents, anti-inflammatories, cytotoxic agents, anti-proliferative agents, vasodilators, gene therapy agents, radioactive agents, immunosuppressants, chemotherapeutics, endothelial cell attractors, endothelial cell promoters, stem cells, hormones, smooth muscle relaxants, mTOR inhibitors and combinations thereof. Often, the therapeutic agent dissolves in a physiological fluid such as blood or cytoplasm.

Sometimes the therapeutic agent is dispersed in a polymeric matrix that is positioned over the polymer anchor coating. Often, the polymeric matrix will diffuse into the polymer anchor coating or bond thereto. In some embodiments, the porosity of the polymer anchor coating may be varied in order to control blending of the polymer matrix with the polymer anchor coating thereby controlling release rate of the therapeutic agent from the polymer matrix. The polymeric matrix may comprise a first polymer layer disposed over the therapeutic agent with an optional second therapeutic agent disposed over the first polymer layer. A second polymer layer may then be placed over the second therapeutic agent. The first and second polymer layers may be adapted to control release rate of the therapeutic agent from the polymer matrix. Often, the polymeric matrix is a different polymer than the polymer anchor coating. Usually, the polymeric matrix biodegrades from the polymer anchor coating over a period not exceeding twenty-four months. The polymeric matrix is usually sufficiently porous or absorptive of a physiological fluid such as blood or cytoplasm to admit the physiological fluid into the polymeric matrix thereby dissolving the therapeutic agent or promoting bioerosion of the polymer matrix.

Possible materials used in the polymer matrix include a material selected from the group consisting of polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride, acrylates, cyanoacrylates, methacrylates and poly(glycerol-sebacate).

These and other embodiments are described in further detail in the following description related to the appended drawing figures.

### BRIEF DESCRIPTION OF THE DRAWINGS

FIG. 1A is a planar view of a stent unrolled and flattened out.

FIG. 1B is a perspective view of the stent illustrated in FIG. 1A.

FIG. 1C is a planar view of the stent illustrated in FIG. 1A after it has been radially expanded.

FIG. 2 shows a plasma chamber where a plasma polymerized tie layer may be applied to a stent.

FIG. 3A shows a schematic diagram of a spray system for applying a therapeutic agent in a polymer matrix to a stent.

FIGS. 3B-3C illustrate exemplary embodiments of a fixture used to hold stents during the spraying process of FIG. 3A.

FIG. 4 illustrates a cross-section of a stent strut having a drug-polymer matrix deposited over a plasma polymerized tie layer that has been applied to the stent surface.

FIGS. 5A-5B illustrate delivery and deployment of a drug coated stent at the target treatment site.

FIG. 6A illustrates a strut of the stent shown in FIGS. 1A-1B.

FIG. 6B illustrates a strut of the stent shown in FIG. 6A after it has been expanded.

FIG. 6C illustrates a strut of the stent shown in FIG. 6A after it has been expanded.

### DETAILED DESCRIPTION OF THE INVENTION

The present invention is of primary interest in connection with medical devices such as stents fabricated from metals and metal alloys. Any of the wide range of metals and alloys known in the art can be used. Examples are the platinum, iridium, titanium, nickel, silver, gold, tantalum, tungsten, alloys of any of the above, Nitinols (a class of shape-memory alloy in which approximately equal proportions of nickel and titanium are the primary constituents), Inconel® (a class of high-strength austenitic nickel-chromium-iron alloys), 300 series stainless steels, magnesium, cobalt, chromium, and cobalt-chromium alloys such as MP35N® (ASTM F562, SPS Technologies, Inc., an alloy of cobalt, chromium, nickel, and molybdenum). The invention also has applicability to stents fabricated from non-metals including both durable and bioerodible polymers or any material for which enhanced adherence characteristics could be beneficial.

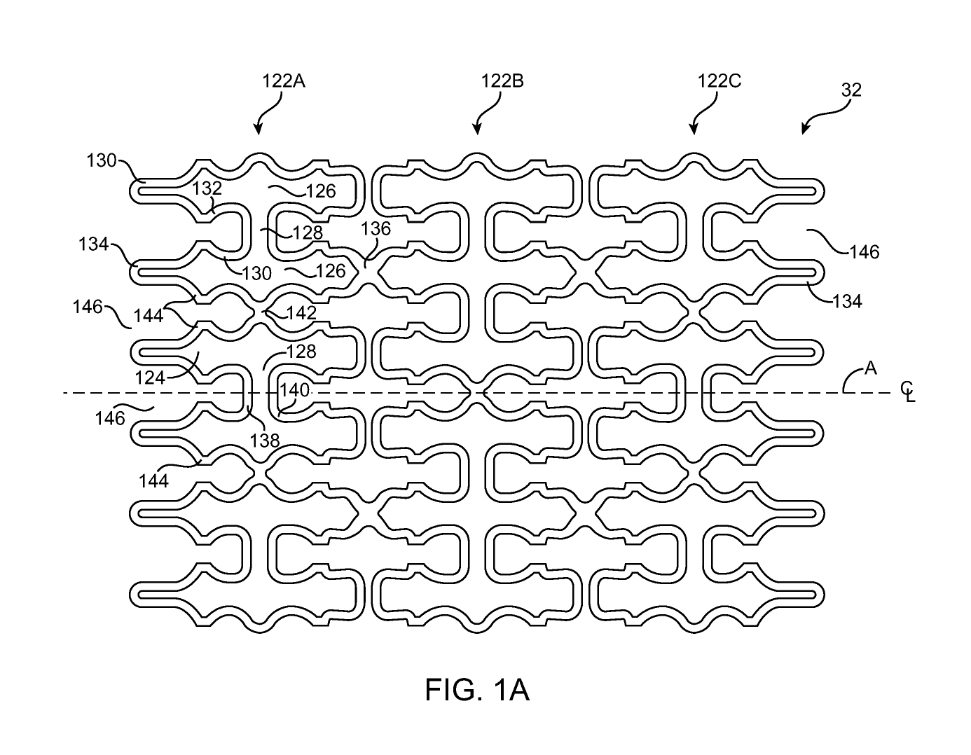

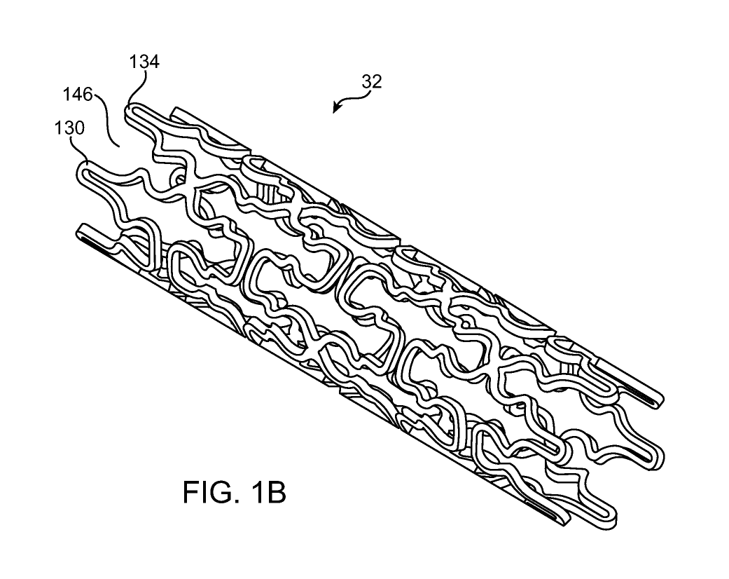

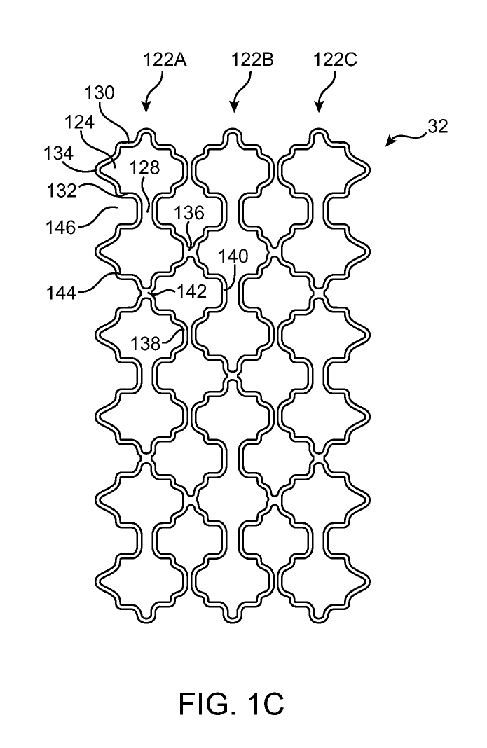

A preferred embodiment of a stent is illustrated in FIGS. 1A-1C. In FIG. 1A a portion of stent segment 32 is shown in a planar shape for clarity. Stent segment 32 comprises parallel rows 122A, 122B and 122C of I-shaped cells 124 formed into a cylindrical shape around axial axis A. FIG. 1B shows the stent of FIG. 1A in perspective view. Referring back to FIG. 1A, cells 124 have upper and lower axial slots 126 and a connecting circumferential slot 128. Upper and lower slots 126 are bounded by upper axial struts 132, lower axial struts 130, curved outer ends 134, and curved inner ends 136. Circumferential slots 128 are bounded by outer circumferential strut 138 and inner circumferential strut 140. Each I-shaped cell 124 is connected to the adjacent I-shaped cell 124 in the same row 122 by a circumferential connecting strut 142. Row 122A is connected to row 122B by the merger or joining of curved inner ends 136 of at least one of upper and lower slots 126 in each cell 124.

In FIGS. 1A and 1B, the stent includes a bulge 144 in upper and lower axial struts 130, 132 extending circumferentially outwardly from axial slots 126. These give axial slots 126 an arrowhead or cross shape at their inner and outer ends. The bulge 144 in each upper axial strut 130 extends toward the bulge 144 in a lower axial strut 132 in the same cell 124 or in an adjacent cell 124, thus creating a concave abutment 146 in the space between each axial slot 126. Concave abutments 146 are configured to receive and engage curved outer ends 134 of cells 124 in the adjacent stent segment, thereby allowing interleaving of adjacent stent segment ends while maintaining spacing between the stent segments. The axial location of bulges 144 along upper and lower axial struts 130, 132 may be selected to provide the desired degree of inter-segment spacing.

FIG. 1C shows stent 32 of FIGS. 1A-1B in an expanded condition, again, unrolled and flattened out for clarity. It may be seen that axial slots 124 are deformed into a circumferentially widened modified diamond shape with bulges 144 on the now diagonal upper and lower axial struts 130, 132. Circumferential slots 128 are generally the same size and shape as in the unexpanded configuration. Bulges 144 have been pulled away from each other to some extent, but still provide a concave abutment 146 to maintain a minimum degree of spacing between adjacent stent segments. As in the earlier embodiment, some axial shortening of each segment occurs upon expansion and stent geometry can be optimized to provide the ideal intersegment spacing.

It should also be noted that the embodiment of FIGS. 1A-1C also enables access to vessel side branches blocked by stent segment 32. Should such side branch access be desired, a dilatation catheter may be inserted into circumferential slot 128 and expanded to provide an enlarged opening through which a side branch may be entered.

A number of other stent geometries are applicable and have been reported in the scientific and patent literature. Other stent geometries include, but are not limited to those disclosed in the following U.S. patents, the full disclosures of which are incorporated herein by reference: U.S. Pat. Nos. 6,315,794; 5,980,552; 5,836,964; 5,527,354; 5,421,955; 4,886,062; and 4,776,337.

Other stents to which the coatings and process of the present invention can be applied are widely disclosed in other publications. In addition to those listed above are the disclosures in U.S. Patent Application Publications Nos. U.S. 2004/0098081 A1 (Landreville, S., et al., published May 20, 2004), US 2005/0149159 A1 (Andreas, B., et al., published Jul. 7, 2005), U.S. 2004/0093061 A1 (Acosta, P., et al., published May 13, 2004), U.S. 2005/0010276 A1 (Acosta, P., et al., published Jan. 13, 2005), U.S. 2005/0038505 A1 (Shulze, J. E., et al., published Feb. 17, 2005), U.S. 2004/0186551 A1 (Kao, S., et al., published Sep. 23, 2004), and U.S. 2003/0135266 A1 (Chew, S., published Jul. 17, 2003). Further disclosures are found in unpublished co-pending U.S. patent application Ser. No. 11/148,713, filed Jun. 8, 2005, entitled “Devices and Methods for Operating and Controlling Interventional Apparatus” (Attorney Docket No. 14592.4002); and Ser. No. 11/148,545, filed Jun. 8, 2005, entitled “Apparatus and Methods for Deployment of Multiple Custom-Length Prosthesis” (Attorney Docket No. 14592.4005). The full disclosures of each of these documents are incorporated herein by reference.

Therapeutic agents, frequently in a polymer matrix, may be deposited onto a stent such as the embodiment illustrated in FIGS. 1A-1B for localized drug delivery. Often, a tie layer is deposited onto the stent first and then the therapeutic agent is deposited onto the tie layer. The tie layer facilitates adhesion between the therapeutic agent and the stent. While various polymers may be used as the tie layer, in the present invention any species that will polymerize in a plasma environment can be deposited in a plasma deposition step onto a stent. Thus plasma polymerization, also known as plasma enhanced chemical vapor deposition (PECVD), may be used to polymerize the tie layer onto a stent surface. This process is distinguished from plasma activation wherein a non-polymerizable gas such as argon, oxygen or nitrogen is used to burn off organic materials from the stent surface and/or leave a highly energized and therefore reactive surface.

As noted above, the selection of the species for plasma polymerization is preferably also coordinated with the selection of the matrix polymer, i.e., the polymeric material deposited in the second step and serving as the carrier for the drug, to achieve compatibility between the two polymers. Alternatively, a mixture of species can be used, where one component of the mixture is compatible with the matrix polymer. The species or mixture to be plasma polymerized will be one that is either in gaseous form under ambient conditions or one that can be readily volatilized. Examples of species that meet this description that may be suitable include but are not limited to unsaturated species such as allyl substituted compounds like allyl alcohol, allyl amine, N-allylmethylamine, allyl chloride, allyl bromide, allyl iodide, allyl acetate, allyl chloroformate, allyl cyanide, allyl cyanoacetate, allyl methyl ether, allyl ethyl ether, allyl propyl ether, allyl isothiocyanate, allyl methacrylate, N-allylurea, N-allylthiourea and allyl trifluoroacetate. Other species that may potentially be used for plasma polymerization include acrylic acid, methacrylic acid, acrylate, methacrylates like 2-hydroxyethylmethacrylate and methacrylate esters. Still other possible species include ethylene glycol, perfluoroalkanes like perfluorocyclohexane, perfluoromethylcyclohexane, perfluoro-1,2-dimethylcyclohexane, perfluoro-1,3-dimethylcyclohexane and perfluoro-1,3,5-trimethylcyclohexane. Yet other species that may potentially be used for plasma polymerization of the tie layer include organosilicones such as trimethylsilane, vinyl trimethylsilane, hexamethyldisiloxane, hexamethyldisilazane. Still other species may include thiophenes, vinyl benzene, and vinyl pyrrolidinone. Further possible examples are saturated species that will fragment in the plasma environment to become free radicals that will readily polymerize. The simplest example is methane; another is perfluoropropane.

The polymer deposited by the plasma process can be continuous over the stent surface or discontinuous, and it can be one that displays engineering properties such as tensile strength and elasticity, or one that does not. The degree of polymerization can vary as well, from polymers that are oligomeric in nature to those of relatively high molecular weight. The plasma-induced polymerization and deposition are achieved by placing the bare stent in contact with the species in gaseous form, preferably in the presence of an inert diluent gas, and imposing high-energy radiation, such as radiofrequency or ultraviolet radiation, sufficient to ionize the species, and the diluent gas when present, to a plasma state. Examples of inert gases that can be used as the diluent gas are argon, helium, and neon. When a diluent is used, the relative amounts of polymerizable species and diluent can vary widely, with species:diluent volumetric ratios preferably ranging from about 10:90 to about 90:10, and most preferably from about 20:80 to about 50:50. The exposure of the stent to the plasma is preferably performed at a reduced pressure in a vacuum chamber, preferably at a pressure of from about 50 mTorr (6.6 Pa) to about 250 mTorr (33 Pa), and most preferably from about 80 mTorr (10.6 Pa) to about 230 mTorr (31 Pa).

Control of the intensity of the plasma treatment to a level that will produce the desired degree of polymerization without excessive crosslinking and thus without depositing a rigid polymer layer on the stent surface can be achieved by limiting the power level, limiting the exposure time, applying the power in a pulsewise manner, controlling gas flow rates or combinations thereof. Pulse may be controlled by adjusting pulse frequency, duty cycle and power. Optimal values of plasma parameters will vary with the chamber size and configuration as well as the electrode design and vacuum pump capacity and conductance. None of these variations are critical to the present invention. In experiments conducted with a Plasma Science PS0500 system having a chamber volume of approximately 5 cubic feet and a plasma work zone of about 2.5 cubic feet, best results were generally achieved with a power level within the range of about 25 Watts to about 1000 Watts, and preferably within the range of about 25 Watts to about 500 Watts. Preferred pressures were generally in the range from about 35 mTorr to about 200 mTorr. Exposure times within the range of about 30 seconds to about 30 minutes, and preferably about 1 minute to about 10 minutes, will likewise produce the best results in most cases. The flow rate of the plasma gas across the stent surface can likewise vary, typically from about 10 to about 1,000 cubic centimeters per minute (measured under, or corrected to, standard temperature and pressure and expressed as sccm), and preferably from about 20 sccm to about 100 sccm. The treatment does not require elevated temperature and is readily performed at temperatures less than 50° C., preferably from about 20° C. to about 40° C. One of ordinary skill in the art will appreciate that temperatures may exceed 50° C. and other operating parameters may exceed the ranges described herein depending on the specific monomers being employed.

As noted above, the thickness of the plasma-deposited polymer need only be great enough to allow the second (matrix) polymer and drug to diffuse into the plasma-deposited polymer during the deposition of the drug and second polymer. Upon contact with a liquid application solution of the second polymer and drug in a carrier solvent, the plasma-deposited polymer may swell to receive the carrier solvent or it may be sufficiently porous independently of any swelling to permit the solvent, second polymer, and drug to diffuse into it. With either mechanism, the plasma-deposited polymer layer will be applied under conditions that result in a coating with a thickness of about 500 Å or less, preferably from about 100 Å to about 500 Å, and most preferably from about 100 Å to about 300 Å, prior to the application of the second polymer and drug. Optionally, the plasma-deposited coating can contain functional groups by which the coating can adhere to second polymer, either by covalent bonds, ionic or Van der Waals attraction or by polar covalent bonding, to further enhance the adhesion of the drug-delivery coating to the stent surface.

The plasma-induced polymerization and deposition can be preceded by cleaning of the stent surface, which can be performed using plasma activation methods. A preliminary plasma treatment can thus be used for sterilization of the stent surface and for removal of contaminants by, for example, etching away weakly bonded molecules. Preliminary plasma treatments can also be used to alter the surface topography of the stent. Examples of gases suitable for these preliminary plasma treatments are molecular oxygen and low molecular weight solvents, such as fluorinated hydrocarbons or carbon tetrafluoride.

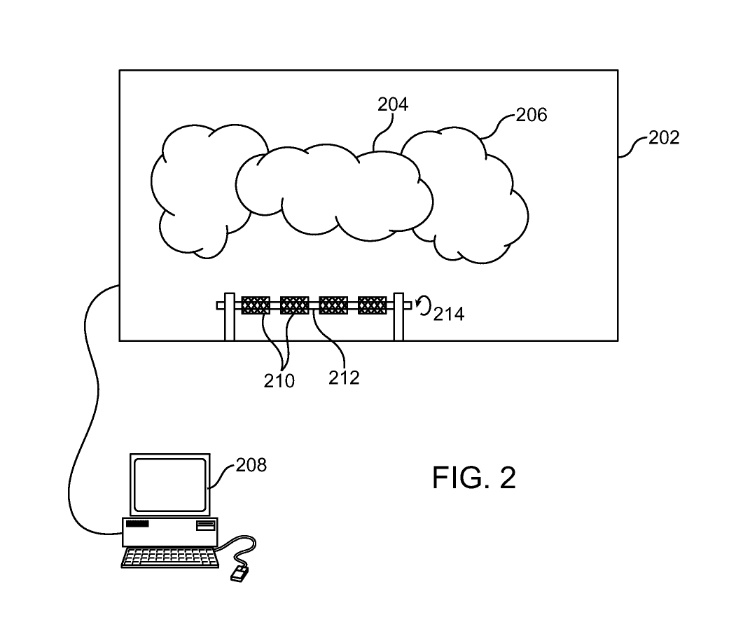

FIG. 2 illustrates a plasma chamber 202 where the plasma polymerized tie layer may be deposited on a stent surface. A plurality of stents 210 are mounted on a mandrel 212 that may rotate 214, although the plasma generally will uniformly contact all surfaces of the stent unless they are masked. Masking of the stent surface using methods well known in the art may be employed to control where the plasma polymerized material is deposited on the stent. The species to be plasma polymerized may be a gas introduced directly into plasma chamber 202 or it may be volatilized 204 and then introduced into the plasma chamber 202. A controller 208 may be used to control the various operating parameter such as power, pulse frequency and exposure time. The process does not typically require elevated temperature and may be conducted at temperatures less than 50° C., preferably from about 20° C. to about 40° C. Additionally, a diluent gas 206, typically a noble gas may also be used during the process.

The second polymer used in the practice of this invention, i.e., the polymer that serves as the primary matrix for the retention and prolonged release of the drug, can be any of the biocompatible and bioerodible polymers known in the art and disclosed in the literature for this use. The terms “erodible” and “bioerodible” are used herein interchangeably to include breakdown of the polymer layer by decomposition, dissolution, or physical separation in the form of fissures and fragmentation, or combinations of these effects. Suitable polymers are those that, once the stent is implanted, will fully dissociate from the stent due to any of these processes over a period of about 2 weeks to about 24 months, preferably from about 2 weeks to about 12 months, and more preferably from about 1 month to about 3 to 9 months. Certain polymers that meet this description are disclosed in Shulze, J. E., et al., U.S. Pat. No. 6,939,376, issued Sep. 6, 2005, and incorporated herein by reference.

Some examples of other biodegradable materials include polyesters such as polyhydroxyalkanoates (PHA) and polyalphahydroxy acids (AHA). Exemplary PHAs include, but are not limited to polymers of 3-hydroxypropionate, 3-hydroxybutyrate, 3-hydroxyvalerate, 3-hydroxycaproate, 3-hydroxyheptanoate, 3-hydroxyoctanoate, 3-hydroxynonanoate, 3-hydroxydecanoate, 3-hydroxyundecanoate, 3-hydroxydodecanoate, 4-hydroxybutyrate and 5-hydroxyvalerate. Examples of AHAs include, but are not limited to various forms of polylactide or polylactic acid including poly(d-lactic acid), poly(1-lactic acid), poly(d,l-lactic acid), polyglycolic acid and polyglycolide, poly(lactic-co-glycolic acid), poly(lactide-co-glycolide), poly(ε-caprolactone) and polydioxanone. Polysaccharides including starch, glycogen, cellulose and chitin may also be used as a biodegradable material. It is also feasible that proteins such as zein, resilin, collagen, gelatin, casein, silk or wool could be used as a biodegradable implant material. Still other materials such as hydrogels including poly(hydroxyethyl methylacrylate), polyethylene glycol, poly(N-isopropylacrylamide), poly(N-vinyl-2-pyrrolidone), cellulose polyvinyl alcohol, silicone hydrogels, polyacrylamides, and polyacrylic acid are potential biodegradable implant materials. Other potential biodegradable materials include lignin, shellac, natural rubber, polyanhydrides, polyamide esters, polyvinyl esters, poly(ethylene vinyl alcohol), polyvinyl alcohol, polyalkylene esters, polyethylene oxide, polyvinylpyrrolidone, polyethylene maleic anhydride and poly(glycerol-sebacate). Other potential materials suitable for the drug matrix may include polycarbonates, polyamides, polyanhydrides, polyamino acids, polyortho esters, polyacetals, degradable polycyanoacrylates, and degradable polyurethanes. Presently preferred are poly(d,l-lactic acid) as the matrix polymer and a polymer obtained by plasma deposition of allyl amine as the plasma-deposited polymer.

The drug can be any of the wide variety of bio-active agents disclosed in the literature for use with stents. Included among these agents are anti-restenosis, anti-proliferative, immunosuppressive, antibiotic, thrombolytic, cytotoxic, and cystostatic agents, as well as growth factors and DNA. Examples of antiproliferative substances are actinomycin D and its derivatives and analogs, angiopeptin, and angiotensin-converting enzyme inhibitors such as captopril, cilazapril and lisinopril. Further examples are calcium channel blockers such as nifedipine and colchicine, fibroblast growth factor (FGF) antagonists, fish oil (omega 3-fatty acid), histamine antagonists, lovastatin, monoclonal antibodies specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, and smooth muscle relaxants such as nitric oxide. Examples of antineoplastics and/or antimitotics are paclitaxel, docetaxel, methotrexate, azathioprine, vincristine, vinblastine, fluorouracil, doxorubicin hydrochloride, and mitomycin. Examples of antiplatelets, anticoagulants, antifibrins, and antithrombins are sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIa platelet membrane receptor antagonist antibody, recombinant hirudin, and thrombin inhibitors such as ANGIOMAX® (Biogen, Inc., Cambridge, Mass., USA). An example of an antiallergic agent is permirolast potassium. A class of particularly preferred therapeutic agents are mTOR inhibitors of which prime examples are rapamycin and its derivatives such as BIOLIMUS A9®, (Biosensors International, Singapore), everolimus, or ABT 578 (Abbott Laboratories, Abbott Park, Ill., USA). Further derivatives of rapamycin that can be used for this purpose are disclosed in Betts, R. E., et al., U.S. Patent Application Publication No. 2005/0131008 A1, published Jun. 16, 2005, the entire contents of which are incorporated herein by reference.

The ratio of therapeutic agent to polymer in the therapeutic agent/matrix application step can vary widely. In some embodiments, this ratio can be as high as 110% therapeutic agent to polymer matrix, while in preferred embodiments, the percentage by weight of therapeutic agent in the polymer matrix ranges from about 0.1% to 50%, preferably from about 0.1% to about 10% and more preferably from about 0.1% to about 1%.

Application of the combination of matrix polymer and drug to the plasma-deposited polymer anchor layer on the stent can be achieved by various methods, some of which are described in the literature for stents bearing therapeutic agents. A preferred method is to form a solution or suspension of the drug and polymer in a volatile liquid solvent or liquid suspending medium, apply the solution or suspension to the stent surface, and then evaporate the solvent or suspending medium. Application can be achieved by dipping, spraying, brush coating, or any equivalent method. A description of spray application is found in Shulze, J. E., et al., U.S. Pat. No. 6,939,376 B2, incorporated herein by reference. Any solvent or suspending medium that will not affect the molecular structure or physical state of the plasma-deposited polymer can be used. Examples of suitable solvents and suspending media are acetone, dichloromethane, and diethyl ether.

In a presently preferred method of application, stents are loaded on a mandrel which can have a circular cross section or a cross section of triangular or other polygonal shape. The mandrel has raised features that engage the inner surface of the stent at discrete locations. These features allow the stent to rotate with the mandrel and also to be removed following the spray operation without damage to the coating. The mandrel is held in a rotary fixture coupled to a computer-controlled rotary stepper motor capable of rotating the mandrel about its longitudinal axis. The motor or mandrel may be mounted on a linear positioning table capable of moving the stent relative to the spray nozzle along at least one horizontal axis.

A mixture of the drug, polymer, and solvent is sprayed onto the mandrel-mounted stents by a spray nozzle mounted on an X-Y-Z positioning system driven by a computer-controlled linear actuator. A pump module supplying the nozzle is connected to a reservoir of solvent and to a reservoir containing the mixture of drug, polymer, and solvent. The system is pressurized with solvent from the solvent reservoir to prevent leaking of the fluid lines and of the reservoir containing the mixture of drug, polymer, and solvent. Preferably, major quantities of the mixture of drug, polymer and solvent are applied to the stent struts at the surfaces of the struts that face radially outward, while a lesser quantity (to produce a coating of lesser thickness) is applied to circumferentially-facing surfaces and to axially-facing sidewalls, and little or no material to surfaces that face radially inward. Much of the solvent in the mixture vaporizes during spraying. Following spraying, the stents are removed from the mandrel and placed in a controlled environment for sufficient time to allow any residual solvent to evaporate. The controlled environment allows operating parameters such as temperature, pressure and gas environment to be regulated. Multiple passes of the spray nozzle over each stent are made until the desired weight or thickness of coating has been applied. Other aspects of suitable stent spraying processes are described in co-pending U.S. patent application Ser. No. 11/099,418, filed Apr. 4, 2005, “Topographic Coatings and Coating Methods for Medical Devices” (Attorney Docket No. 021629-002610US), the contents of which are incorporated herein by reference.

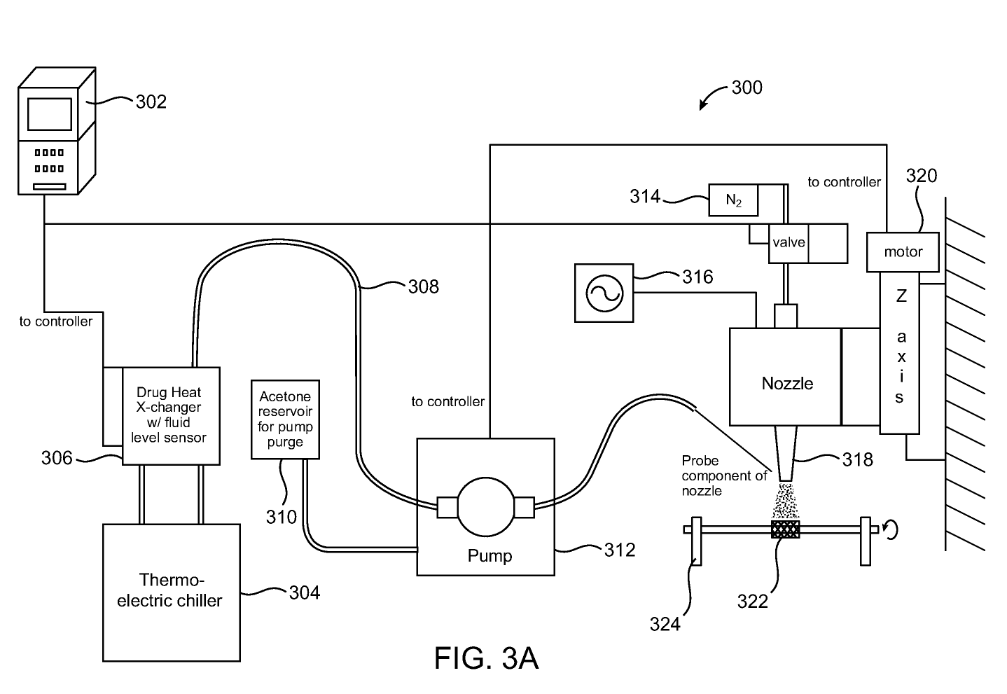

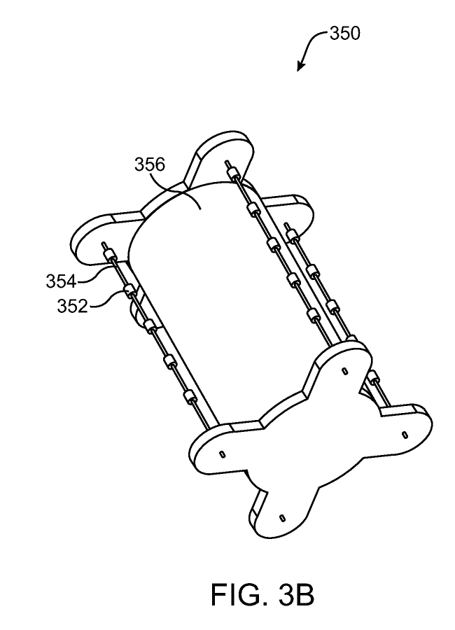

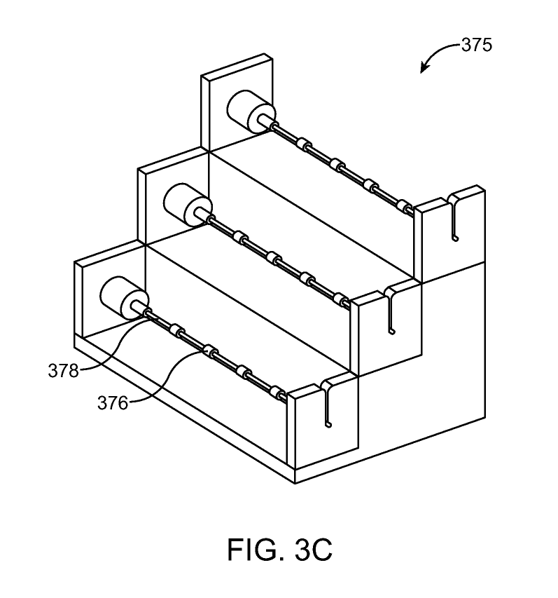

FIG. 3A shows a schematic diagram of a system 300 for coating a stent with a therapeutic agent. Coating system 300 includes a controller 302 that allows all process parameters of the system 300 to be pre-programmed or manually selected, including controlling temperatures, pressures, positions, etc. A reservoir 306 holds the therapeutic agent and a polymer, such as Biolimus A9™ and PLA, dissolved in a solvent such as acetone. Chiller 304 allows the temperature of reservoir 306 to be controlled so as to prevent degradation of the therapeutic agent or excessive solvent evaporation. A pump 312, such as an IVEK pump, pumps the fluid containing the therapeutic agent and polymer through piping 308 to the spray nozzle 318, such as a Sono-Tek Micromist nozzle, where it can be deposited over a stent surface, 322. A second reservoir 310 may also contain acetone or another solvent to help clean and purge the system as needed. Inert gas 314 such as nitrogen may also be used to pressurize the system 300 thereby directing the fluid to the stent. A broadband generator 316 is also used in the system in order to volatilize the therapeutic agent and polymer to facilitate spraying it on the stent 322. The spray nozzle 318 may also be coupled to an XYZ positioning system so as to allow precise movement of the nozzle 318 with respect to the stent 322. In spray system 300, a single stent 322 is shown mounted to a rotating mandrel 324. Multiple stents may be loaded onto the mandrel and a positioning system may also be used to move the stent with respect to the spray nozzle 318. This way, a uniform coating of therapeutic agent and polymer matrix may be applied to the stent surface.

One will of course appreciate that many other fixtures may be used to hold and position stents during the spraying process. For example, in FIG. 3B, fixture 350 accommodates multiple stents 352 on each rotating mandrel 354 and a plurality of mandrels are circumferentially disposed around a rotating drum 356, thereby increasing the stent processing capacity. Another exemplary embodiment of a spray fixture is seen in the perspective view of FIG. 3C. In FIG. 3C, multiple stents 376 are mounted on rotating mandrels 378, arranged in a step-wise fashion in the fixture.

FIG. 4 shows a cross section of a stent strut 402 after the plasma polymerized tie layer and drug-polymer matrix have been applied. A plasma polymerized, ultra thin, monomolecular tie layer 404 is first applied to the stent surfaces as described above. The tie layer 404 is fairly uniform thickness on all stent surfaces. The polymer matrix 406 is then coated over the tie layer 404. The polymer matrix contains a drug 408 dispersed therein. The spray process described above typically results in a thicker coating on the top surface 410 of the stent, with a thinner coating on the stent sides 412 and an even thinner coating on the stent bottom surface 414. However, one should appreciate that the spray coating may be adjusted to control these thicknesses.

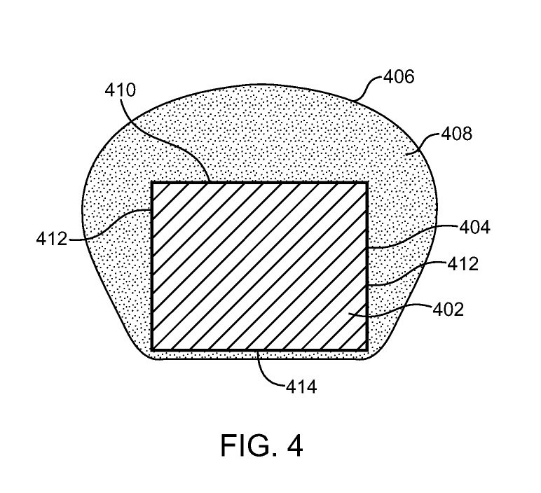

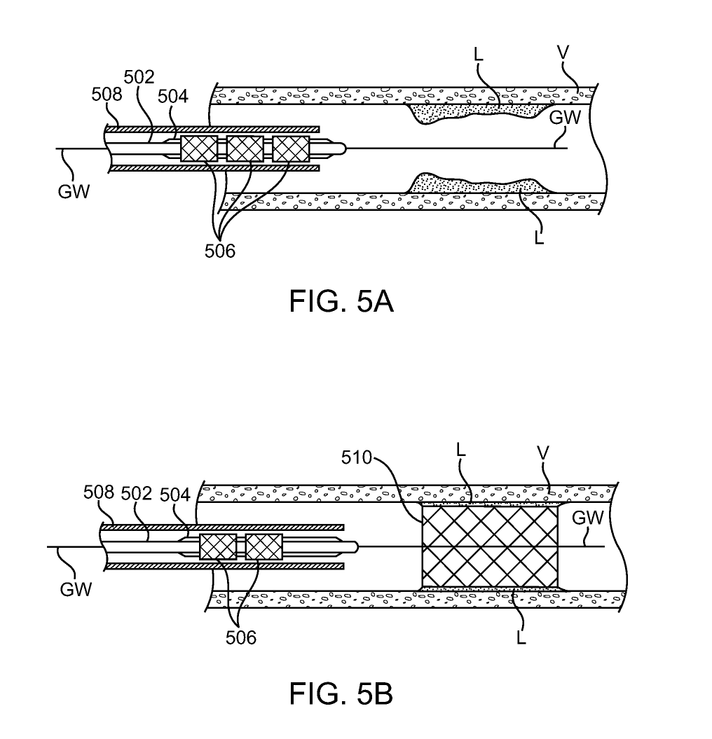

Once the stents have been coated with a drug, they may be loaded onto a delivery catheter and delivered to a target treatment site. FIGS. 5A-5B illustrate an exemplary embodiment of delivery and deployment of a drug eluting stent. In FIG. 5A, standard catheterization techniques are used to introduce a delivery catheter 502 into a coronary artery. Delivery catheter 502 is advanced over a guidewire GW in the coronary artery V having a stenotic lesion L. In this exemplary embodiment, a plurality of stents 506 are disposed over a balloon 504 which is coupled to the delivery catheter 502 near its distal end. A sheath 508 is disposed over the stents 506 in order to protect them during delivery. In FIG. 5B, a single stent 510 is deployed into the lesion L and the delivery catheter is retracted away from the lesion L. The stent 510 now provides mechanical scaffolding to help keep the coronary artery patent and the drug coating can elute into treatment region in order to prevent restenosis. FIGS. 5A-5B show deployment of a single fixed length stent to treat a lesion. In some situations, it is advantageous to be able to customize stent length in situ in order to more accurately match stent length to lesion length. The use of multiple stent segments has been proposed to allow customization of stent length as well as treatment of treatment of multiple lesions. U.S. Patent Publication No. 2007/0027521, entitled “Apparatus and Methods for Deployment of Multiple Custom-Length Prostheses” discloses such a method and the entire contents are incorporated herein by reference. Stents coated with a therapeutic agent as described herein may be delivered using the apparatus and methods described in the aforementioned publication thereby allowing stent length to be customized in situ.

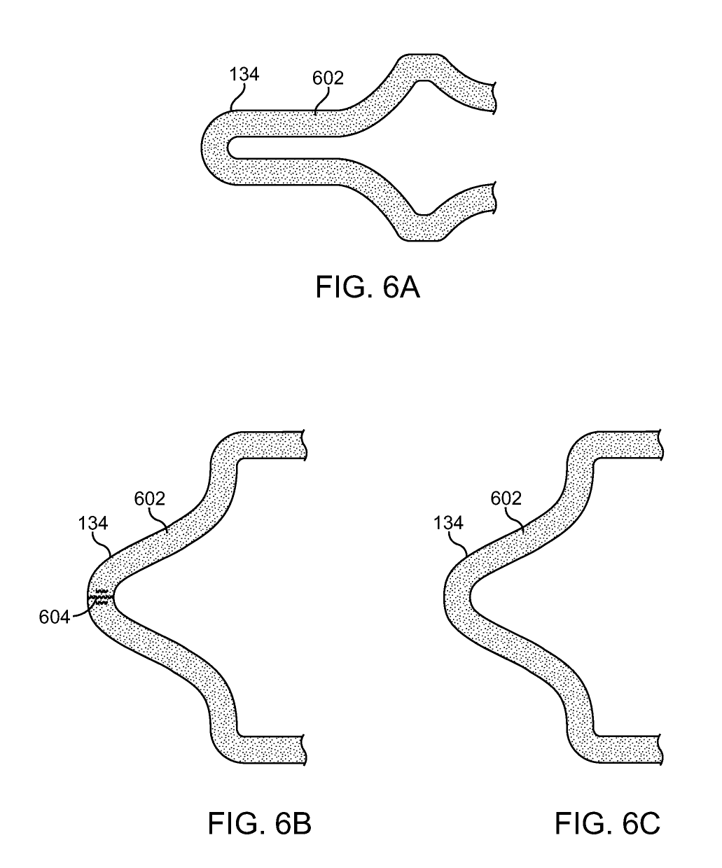

Portions of stent struts experience high stress and strain during deployment of the stent. For example, FIG. 6A illustrates an unexpanded stent strut 134 having a drug-polymer matrix coating 602 disposed thereon. FIG. 6B shows the same strut 134 after the stent has been expanded. Often with traditional drug coatings, cracking 604 results in the high strain regions of the stent during expansion. Strain can result in delamination of the drug coating from the stent and therefore is undesirable. However, in the present invention, the plasma polymerized tie layer is non-rigid and hence is able to flex with the strut as it expands thereby avoiding cracking and delamination. Other strained regions of the stent may also result in cracking of the tie layer, such as the inner circumferential struts 140 of FIG. 1A. FIG. 6C shows stent strut 134 in the expanded state with no cracks in the drug coating after it has been applied along with a plasma polymerized tie layer according to the methods described herein. Also, in some delivery systems, the stent may be abraded during delivery, resulting in delamination of the drug coating. The polymer anchor layer helps the drug coating to adhere to the stent even under abrasion.

The following examples illustrate various aspects of fabrication and use of a stent having a plasma polymerized anchor coating with a therapeutic agent disposed thereon according to the methods disclosed herein. These examples are not intended to limit the scope of the present invention.

### Example 1

Cobalt-chromium alloy stents were loaded onto a mandrel and placed into a holding fixture within a Plasma Science PS0500 plasma chamber. A vacuum was drawn inside the chamber and surface cleaning of the stents was performed by plasma treating the stents with oxygen. Next, allyl amine was plasma polymerized onto the stent surface followed by quenching and purging in argon gas. The stents were removed from the plasma chamber and a therapeutic agent, a matrix of Biolimus A9 and polylactide (PLA) in a solvent (acetone) was then sprayed on the plasma polymerized stents. After spraying, the stents were transferred to a vacuum chamber to evaporate the solvent. The therapeutic agent coating was then evaluated by a series of mechanical tests such as scratch testing, followed by visual inspection. Test results demonstrated that the therapeutic agent adhered to the stent and coating integrity was comparable to control stents having a Biolimus A9/PLA matrix deposited over a parylene primer layer that had been applied to the stent using chemical vapor deposition (CVD).

### Example 2

Cobalt-chromium stents were cleaned similarly as above with oxygen. The flow rate for the gas was 350 sccm, and the power was 450 Watts for 5 minutes. Allyl amine or acrylic acid was then plasma polymerized onto the stent surface using a flow rate of 7 ml/hour, at 60% to 80% power (300-400 Watts) for two minutes, followed by quenching and purging under three, one-minute argon gas purges. Biolimus A9/PLA was then sprayed onto the plasma polymer coating as previously described. The coated stents were then terminally sterilized by irradiation with a minimum of 25 kGy. Coated stents were also placed under accelerated aging conditions (approximately 40° C. for ten days) and then crimped onto delivery catheters for deployment. Drug elution testing demonstrated similar elution rates for both the plasma polymerized stents as well as the control samples which had Biolimus A9/PLA deposited over a parylene primer layer deposited using CVD. Coating integrity for the plasma polymerized stents after deployment demonstrated that the coating remained coupled to the deployed stent and test results were comparable to the parylene control group. Similarly 7 day and 28 day animal implant results measured the percent stenosis after implantation into a coronary artery with similar stenosis rates for both the plasma polymerized stents as well as the parylene control stents. Furthermore, biocompatibility testing of the plasma polymerized stents demonstrated that the test stents were non-cytotoxic using an MEM elution as well as non-hemolytic. The plasma polymerization method therefore is a feasible method of coupling a therapeutic agent to a metal stent.

While the exemplary embodiments have been described in some details for clarity of understanding and by way of example, a variety of additional modifications, adaptations and changes may be clear to those of skill in the art. Hence, the scope of the present invention is limited solely by the appended claims.

What is claimed is:

1. A method for the manufacture of an intraluminal device bearing a therapeutic agent releasable from the device in a time-controlled manner, the method comprising:
exposing a metallic substrate to a gaseous plasma form of a substance that polymerizes in the plasma form under conditions causing the substance to form a polymer anchor coating of about 500 Å in thickness or less on the substrate; and
depositing over the polymer anchor coating a layer containing the therapeutic agent wherein substantially all of the therapeutic agent is releasable into a physiological environment gradually over a period ranging from about one hour up to about six months.

2. A method as in claim 1, wherein the polymer anchor coating is adapted to withstand significant cracking during expansion of the intraluminal device.

3. A method as in claim 1, wherein the polymer anchor coating remains coupled to the intraluminal device during expansion thereof, without substantially separating therefrom.

4. A method as in claim 1, wherein a physiological fluid dissolves the therapeutic agent.

5. A method as in claim 4, wherein the physiological fluid comprises blood or cytoplasm.

6. A method as in claim 1, wherein the step of depositing results in swelling of the polymer anchor coating thereby enhancing diffusion of the therapeutic agent into the polymer anchor coating.

7. A method as in claim 1, wherein the metallic substrate comprises a material selected from the group consisting of stainless steel, nickel-titanium alloys and cobalt-chromium alloys.

8. A method as in claim 1, wherein the substance is either in gaseous form under ambient conditions or the substance can be volatilized.

9. A method as in claim 8, wherein the substance comprises a material selected from the group consisting of allyl substituted compounds, acrylic acids, methacrylic acids, acrylates, methacrylates, ethylene glycol, organosilicones, thiophenes, vinyl benzene, vinyl pyrrolidinone, and methane.

10. A method as in claim 1, wherein the polymer anchor coating is continuous over substantially all of a surface of the metallic substrate.

11. A method as in claim 1, wherein the step of exposing the metallic substrate comprises exposing the metallic substrate to a inert diluent noble gas in the presence of the substance to be polymerized.

12. A method as in claim 1, further comprising masking a portion of the substrate so as to selectively apply the polymer anchor coating to the substrate.

13. A method as in claim 1, further comprising controlling the degree of polymerization of the substance.

14. A method as in claim 13, wherein controlling comprises a step selected from the group consisting of limiting power level, limiting exposure time and applying power in a pulsewise manner.

15. A method as in claim 1, further comprising controlling the degree of cross-linking of the substance.

16. A method as in claim 15, wherein controlling comprises a step selected from the group consisting of limiting power level, limiting exposure time and applying power in a pulsewise manner.

17. A method as in claim 1, further comprising cleaning of a surface of the substrate.

18. A method as in claim 1, wherein the therapeutic agent comprises at least one of antibiotics, thrombolytics, anti-platelet agents, anti-inflammatories, cytotoxic agents, anti-proliferative agents, vasodilators, gene therapy agents, radioactive agents, immunosuppressants, chemotherapeutics, endothelial cell attractors, endothelial cell promoters, stem cells, hormones, smooth muscle relaxants, mTOR inhibitors and combinations thereof.

19. A method as in claim 1, wherein the step of depositing comprises one of dipping, spraying, brush coating, syringe deposition, chemical vapor deposition or plasma deposition of the layer of the therapeutic agent over the polymer anchor coating.

20. A method as in claim 1, wherein the step of depositing comprises rotating a mandrel with the intraluminal device disposed thereon.

21. A method as in claim 1, wherein the therapeutic agent is dispersed in a polymeric matrix positioned over the polymer anchor coating.

22. A method as in claim 21, wherein the polymeric matrix comprises a first polymer layer disposed over the therapeutic agent.

23. A method as in claim 22, wherein the polymeric matrix is adapted to control release rate of the therapeutic agent from the layer containing the therapeutic agent.

24. A method as in claim 22, wherein the polymeric matrix further comprises a second therapeutic agent disposed over the first polymer layer.

25. A method as in claim 24, wherein the polymeric matrix further comprises a second polymer layer disposed over the second therapeutic agent.

26. A method as in claim 21, wherein the polymeric matrix is a different polymer than the polymer anchor coating.

27. A method as in claim 21, wherein the polymeric matrix biodegrades from the polymer anchor coating over a period not exceeding twenty-four months.

28. A method as in claim 21, wherein the polymeric matrix diffuses into the polymer anchor coating.

29. A method as in claim 21, wherein the polymeric matrix bonds to the polymer anchor coating.

30. A method as in claim 21, wherein the polymeric matrix is sufficiently porous or absorptive of a physiological fluid to admit the physiological fluid into the polymeric matrix thereby dissolving the therapeutic agent.

31. A method as in claim 30, wherein the physiological fluid comprises blood or cytoplasm.

32. A method as in claim 21, wherein the polymeric matrix is sufficiently porous or absorptive of a physiological fluid to admit the physiological fluid into the polymeric matrix, thereby promoting bioerosion of the matrix.

33. A method as in claim 32, wherein the physiological fluid comprises blood or cytoplasm.

34. A method as in claim 21, wherein the polymer matrix comprises a material selected from the group consisting of polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride, acrylates, cyanoacrylates, methacrylates and poly(glycerol-sebacate).

35. A method as in claim 21, further comprising varying porosity of the polymer anchor coating in order to control blending of the polymer matrix with the polymer anchor coating thereby controlling release rate of the therapeutic agent from the polymer matrix.

36. A method for the manufacture of an intraluminal device bearing a therapeutic agent releasable from the device in a time-controlled manner, the method comprising:
exposing a metallic substrate to a gaseous plasma form of a substance that polymerizes in the plasma form under conditions causing the substance to form a polymer anchor coating on the substrate; and
depositing over the polymer anchor coating a layer containing the therapeutic agent in a polymer matrix that releases substantially all of the therapeutic agent into a physiological environment gradually over a period ranging from about one hour up to about six months,
and wherein following release of the therapeutic agent, any polymer remaining on the substrate is about 500 Å or less in thickness.

37. A method as in claim 36, wherein the polymer anchor coating is adapted to withstand significant cracking during expansion of the intraluminal device.

38. A method as in claim 36, wherein the polymer anchor coating remains coupled to the intraluminal device during expansion thereof, without substantially separating therefrom.

39. A method as in claim 36, wherein a physiological fluid dissolves the therapeutic agent.

40. A method as in claim 39, wherein the physiological fluid comprises blood or cytoplasm.

41. A method as in claim 36, wherein the step of depositing results in swelling of the polymer anchor coating thereby enhancing diffusion of the therapeutic agent into the polymer anchor coating.

42. A method as in claim 36, wherein the metallic substrate comprises a material selected from the group consisting of stainless steel, nickel-titanium alloys and cobalt-chromium alloys.

43. A method as in claim 36, wherein the substance is either in gaseous form under ambient conditions or the substance can be volatilized.

44. A method as in claim 43, wherein the substance comprises a material selected from the group consisting of allyl substituted compounds, acrylic acids, methacrylic acids, acrylates, methacrylates, ethylene glycol, organosilicones, thiophenes, vinyl benzene, vinyl pyrrolidinone, and methane.

45. A method as in claim 36, wherein the polymer anchor coating is continuous over substantially all of a surface of the metallic substrate.

46. A method as in claim 36, wherein the step of exposing the metallic substrate comprises exposing the metallic substrate to a inert diluent noble gas in the presence of the substance to be polymerized.

47. A method as in claim 36, further comprising masking a portion of the substrate so as to selectively apply the polymer anchor coating to the substrate.

48. A method as in claim 36, further comprising controlling the degree of polymerization of the substance.

49. A method as in claim 48, wherein controlling comprises a step selected from the group consisting of limiting power level, limiting exposure time and applying power in a pulsewise manner.

50. A method as in claim 36, further comprising controlling the degree of cross-linking of the substance.

51. A method as in claim 50, wherein controlling comprises a step selected from the group consisting of limiting power level, limiting exposure time and applying power in a pulsewise manner.

52. A method as in claim 36, further comprising cleaning of a surface of the substrate.

53. A method as in claim 36, wherein the therapeutic agent comprises at least one of antibiotics, thrombolytics, anti-platelet agents, anti-inflammatories, cytotoxic agents, anti-proliferative agents, vasodilators, gene therapy agents, radioactive agents, immunosuppressants, chemotherapeutics, endothelial cell attractors, endothelial cell promoters, stem cells, hormones, smooth muscle relaxants, mTOR inhibitors and combinations thereof.

54. A method as in claim 36, wherein the step of depositing comprises one of dipping, spraying, brush coating, syringe deposition, chemical vapor deposition or plasma deposition of the solid layer of the therapeutic agent over the polymer anchor coating.

55. A method as in claim 36, wherein the step of depositing comprises rotating a mandrel with the intraluminal device disposed thereon.

56. A method as in claim 36, wherein the polymeric matrix is a different polymer than the polymer anchor coating.

57. A method as in claim 36, wherein the polymeric matrix biodegrades from the polymer anchor coating over a period not exceeding twenty-four months.

58. A method as in claim 36, wherein the polymeric matrix comprises a first polymer layer disposed over the therapeutic agent.

59. A method as in claim 58, wherein the first layer is adapted to control release rate of the therapeutic agent from the polymeric matrix.

60. A method as in claim 58, wherein the polymeric matrix further comprises a second therapeutic agent disposed over the first polymer layer.

61. A method as in claim 60, wherein the polymeric matrix further comprises a second polymer layer disposed over the second therapeutic agent.

62. A method as in claim 36, wherein the polymeric matrix diffuses into the polymer anchor coating.

63. A method as in claim 36, wherein the polymeric matrix bonds to the polymer anchor coating.

64. A method as in claim 36, wherein the polymeric matrix is sufficiently porous or absorptive of a physiological fluid to admit the physiological fluid into the polymeric matrix thereby dissolving the therapeutic agent.

65. A method as in claim 64, wherein the physiological fluid comprises blood or cytoplasm.

66. A method as in claim 36, wherein the polymeric matrix is sufficiently porous or absorptive of a physiological fluid to admit the physiological fluid into the polymeric matrix, thereby promoting bioerosion of the matrix.

67. A method as in claim 66, wherein the physiological fluid comprises blood or cytoplasm.

68. A method as in claim 36, wherein the polymer matrix comprises a material selected from the group consisting of polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride, acrylates, cyanoacrylates, methacrylates and poly(glycerol-sebacate).

69. A method as in claim 36, further comprising varying porosity of the polymer anchor coating in order to control blending of the polymer matrix with the polymer anchor coating thereby controlling release rate of the therapeutic agent from the polymer matrix.

70. A stent for placement in a body lumen, the stent comprising:
a plurality of struts coupled together forming a substantially tubular structure, the plurality of struts having a polymer anchor coating of about 500 Å in thickness or less disposed thereon and a layer containing a therapeutic agent positioned over the polymer anchor coating, wherein the polymer anchor coating is formed from a gaseous plasma form of a substance that polymerizes on the struts while in the plasma form, and
wherein substantially all of the therapeutic agent is released into a physiological environment gradually over a period ranging from about one hour up to about six months.

71. A stent as in claim 70, wherein the tubular structure is self-expanding.

72. A stent as in claim 70, wherein the tubular structure is balloon expandable.

73. A stent as in claim 70, wherein the polymer anchor coating is adapted to withstand significant cracking during expansion of the stent.

74. A stent as in claim 70, wherein the polymer anchor coating remains coupled to the intraluminal device during expansion thereof, without substantially separating therefrom.

75. A stent as in claim 70, wherein a physiological fluid dissolves the therapeutic agent.

76. A stent as in claim 75, wherein the physiological fluid comprises blood or cytoplasm.

77. A stent as in claim 70, wherein the polymer anchor coating swells upon contact with the therapeutic agent thereby enhancing diffusion of the therapeutic agent into the polymer anchor coating.

78. A stent as in claim 70, wherein the struts are metal.

79. A stent as in claim 78, wherein the plurality of struts comprise a material selected from the group consisting of stainless steel, nickel-titanium alloys and cobalt-chromium alloys.

80. A stent as in claim 70, wherein the struts are a polymer.

81. A stent as in claim 70, wherein the struts are at least partially bioerodible.

82. A stent as in claim 70, wherein the substance is either in gaseous form under ambient conditions or the substance can be volatilized.

83. A stent as in claim 82, wherein the substance comprises a material selected from the group consisting of allyl substituted compounds, acrylic acids, methacrylic acids, acrylates, methacrylates, ethylene glycol, organosilicones, thiophenes, vinyl benzene, vinyl pyrrolidinone, and methane.

84. A stent as in claim 70, wherein the therapeutic agent inhibits restenosis.

85. A stent as in claim 70, wherein the therapeutic agent comprises at least one of antibiotics, thrombolytics, anti-platelet agents, anti-inflammatories, cytotoxic agents, anti-proliferative agents, vasodilators, gene therapy agents, radioactive agents, immunosuppressants, chemotherapeutics, endothelial cell attractors, endothelial cell promoters, stem cells, hormones, smooth muscle relaxants, mTOR inhibitors and combinations thereof.

86. A stent as in claim 70, wherein the polymer anchor coating is continuous over substantially all of a surface of at least one of the struts.

87. A stent as in claim 70, wherein the therapeutic agent is dispersed in a polymeric matrix positioned over the polymer anchor coating.

88. A stent as in claim 70, wherein the polymeric matrix comprises a first polymer layer disposed over the therapeutic agent.

89. A method as in claim 88, wherein the first layer is adapted to control release rate of the therapeutic agent from the polymeric matrix.

90. A method as in claim 88, wherein the polymeric matrix further comprises a second therapeutic agent disposed over the first polymer layer.

91. A method as in claim 60, wherein the polymeric matrix further comprises a second polymer layer disposed over the second therapeutic agent.

92. A stent as in claim 87, wherein the polymeric matrix is a different polymer than the polymer anchor coating.

93. A stent as in claim 87, wherein the polymeric matrix biodegrades from the polymer anchor coating over a period not exceeding twenty-four months.

94. A stent as in claim 87, wherein the polymeric matrix diffuses into the polymer anchor coating.

95. A stent as in claim 87, wherein the polymeric matrix bonds to the polymer anchor coating.

96. A stent as in claim 87, wherein the polymeric matrix is sufficiently porous or absorptive of a physiological fluid to admit the fluid into the polymeric matrix thereby dissolving the therapeutic agent.

97. A stent as in claim 96, wherein the physiological fluid comprises blood or cytoplasm.

98. A stent as in claim 87, wherein the polymeric matrix is sufficiently porous or absorptive of a physiological fluid to admit the fluid into the polymeric matrix thereby promoting bioerosion of the polymer matrix.

99. A stent as in claim 98, wherein the physiological fluid comprises blood or cytoplasm.

100. A stent as in claim 87, wherein the polymer anchor coating swells upon contact with the polymeric matrix thereby enhancing diffusion of the polymeric matrix into the polymer anchor coating.

101. A stent as in claim 87, wherein the polymer matrix comprises a material selected from the group consisting of polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride, acrylates, cyanoacrylates, methacrylates and poly(glycerol-sebacate).

102. A method for delivering a therapeutic agent to a target treatment site, the method comprising:
introducing a delivery catheter having a stent disposed thereon to the target treatment site; and
deploying the stent into the target treatment site,
wherein the stent comprises a plurality of struts having a polymer anchor coating of about 500 Å in thickness or less disposed thereon and a layer containing the therapeutic agent positioned over the polymer anchor coating, wherein the polymer anchor coating is formed from a gaseous plasma form of a substance that polymerizes on the struts while in the plasma form, and
wherein substantially all of the therapeutic agent is released into the target treatment site gradually over a period ranging from about one hour up to about 6 months.

103. A method as in claim 102, wherein the therapeutic agent inhibits restenosis in a blood vessel following release of the therapeutic agent.

104. A method as in claim 102, wherein deploying the stent comprises deploying the stent into an artery.

105. A method as in claim 102, wherein the artery is a coronary artery or a peripheral artery.

106. A method as in claim 102, wherein deploying the stent comprises radially expanding the stent.

107. A method as in claim 106, wherein the stent is self-expanding.

108. A method as in claim 106, wherein deploying the stent comprises expanding a balloon.

109. A method as in claim 102, wherein deploying comprises radially expanding the stent without significant cracking of the polymer anchor coating.

110. A method as in claim 102, wherein deploying comprises radially expanding the stent without substantially separating the polymer anchor coating from the stent.

111. A method as in claim 102, wherein the polymer anchor coating swells upon contact with the therapeutic agent thereby enhancing diffusion of the therapeutic agent into the polymer anchor coating.

112. A method as in claim 102, wherein the substance is either in gaseous form under ambient conditions or the substance can be volatilized.

113. A method as in claim 112, wherein the substance comprises a material selected from the group consisting of allyl substituted compounds, acrylic acids, methacrylic acids, acrylates, methacrylates, ethylene glycol, organosilicones, thiophenes, vinyl benzene, vinyl pyrrolidinone, and methane.

114. A method as in claim 102, wherein the polymer anchor coating is continuous over substantially all of a surface of the struts.

115. A method as in claim 102, wherein the therapeutic agent comprises at least one of antibiotics, thrombolytics, anti-platelet agents, anti-inflammatories, cytotoxic agents, anti-proliferative agents, vasodilators, gene therapy agents, radioactive agents, immunosuppressants, chemotherapeutics, endothelial cell attractors, endothelial cell promoters, stem cells, hormones, smooth muscle relaxants, mTOR inhibitors and combinations thereof.

116. A method as in claim 102, wherein the therapeutic agent is dispersed in a polymeric matrix positioned over the polymer anchor coating.

117. A stent as in claim 116, wherein the polymeric matrix comprises a first polymer layer disposed over the therapeutic agent.

118. A method as in claim 117, wherein the first layer is adapted to control release rate of the therapeutic agent from the polymeric matrix.

119. A method as in claim 117, wherein the polymeric matrix further comprises a second therapeutic agent disposed over the first polymer layer.

120. A method as in claim 119, wherein the polymeric matrix further comprises a second polymer layer disposed over the second therapeutic agent.

121. A method as in claim 116, wherein the polymeric matrix is a different polymer than the polymer anchor coating.

122. A method as in claim 116, wherein the polymeric matrix biodegrades from the polymer anchor coating over a period not exceeding twenty-four months.

123. A method as in claim 116, wherein the polymeric matrix diffuses into the polymer anchor coating.

124. A method as in claim 116, wherein the polymeric matrix bonds to the polymer anchor coating.

125. A method as in claim 116, wherein the polymeric matrix is sufficiently porous or absorptive of a physiological fluid to admit the fluid into the polymeric matrix thereby dissolving the therapeutic agent.

126. A method as in claim 125, wherein the physiological fluid comprises blood or cytoplasm.

127. A method as in claim 116, wherein the polymeric matrix is sufficiently porous or absorptive of a physiological fluid to admit the fluid into the polymeric matrix thereby promoting bioerosion of the polymer matrix.

128. A method as in claim 127, wherein the physiological fluid comprises blood or cytoplasm.

129. A method as in claim 116, wherein the polymer anchor coating swells upon contact with the polymeric matrix thereby enhancing diffusion of the polymeric matrix into the polymer anchor coating.

130. A method as in claim 116, wherein the polymer matrix comprises a material selected from the group consisting of polyhydroxyalkanoates, polyalphahydroxy acids, polysaccharides, proteins, hydrogels, lignin, shellac, natural rubber, polyanhydrides, polyamide esters, polyvinyl esters, polyvinyl alcohols, polyalkylene esters, polyethylene oxide, polyvinylpyrrolidone, polyethylene maleic anhydride, acrylates, cyanoacrylates, methacrylates and poly(glycerol-sebacate).
